(19) United States
(12) Patent Application Publication
(10) Pub. No.: US 2020/0372993 A1
(43) Pub. Date: Nov. 26, 2020

(54) SYSTEMS, METHODS, AND APPARATUS FOR TAILORED DOSING OF CANNABIS FORMULATIONS

(71) Applicant: HEY MARY, LLC., Brooklyn, NY (US)

(72) Inventor: Paul Chu, Brooklyn, NY (US)

(21) Appl. No.: 16/759,326

(22) PCT Filed: Oct. 27, 2018
(86) PCT No.: PCT/US18/57890

§ 371 Date: Apr. 25, 2020

Related U.S. Application Data
(60) Provisional application No. 62/577,891, filed on Oct. 27, 2017.

(51) Int. Cl. G16H 20/13; G16H 80/00; G16H 10/60; G16H 40/63; G06N 20/00; A61K 36/185

(52) U.S. Cl. CPC G16H 20/13; G16H 80/00; A61K 36/185; G16H 40/63; G06N 20/00; G16H 10/60

(57) ABSTRACT

Disclosed herein are systems, methods, and apparatus for tailoring dosages of compounds to a user's individual needs using a software application comprising artificial intelligence and hardware for formulating and dispensing the tailored dosages determined from user information and available databases.

### CROSS-REFERENCE TO RELATED APPLICATIONS

This application is a U.S. National Stage Application, filed under 35 U.S.C. § 371, of International Application No. PCT/US2018/057890, filed Oct. 27, 2018, which claims priority benefit of U.S. Provisional Patent Application Ser. No. 62/577,891, filed Oct. 27, 2017, and entitled “Systems, Methods, Devices, and Apparatus for Cannabis Formulations.” The disclosures of the aforementioned Provisional Patent Application Ser. No. 62/577,891 and International Application No. PCT/US2018/057890 are hereby incorporated by reference in their entirety.

### BACKGROUND

In recent years there is an increasing interest in bespoke services and product lines and crafting products towards individual tastes and needs. Cannabis products have been consumed in various forms for thousands of years for therapeutic and general wellness uses. We are currently seeing a renaissance in our recognition of the value that can be brought to human civilization by cultivating and ingesting the flowering plant from the genus Cannabis.

### SUMMARY

At this time of great interest in the benefits of Cannabis, Cannabis has not yet realized its full value. The inventor has realized that the benefits of Cannabis compounds have been hindered because of the problems of inconsistent and inaccurate dosing and lack of tailoring of Cannabis formulations to the needs of users because of the inherent variability of users in their sensitivity and receptivity to different Cannabis compounds and lack of correlation of available data on Cannabis compounds with user needs. The inventor solves these problems in the art with the present disclosure by providing systems, methods, and apparatus that can determine formulations of Cannabis compounds tailored to the needs of users and provide for consistent and accurate dosing with these formulations. The systems, methods, and apparatus described herein provide for simple, efficient, and convenient development of tailored Cannabis formulations for individual users to meet their individual needs and goals.

The precision dosing, as described herein, enables users to take advantage of the full spectrum of beneficial properties of Cannabis compounds for all wellness, therapeutic, and recreational uses in which data is or becomes available. And, the systems, methods, and apparatus described herein enable the improvement in tailored formulations as we obtain better data about Cannabis uses and better understanding of the human body. The methods described herein can generate better therapeutic formulations and/or solutions to physical and psychological needs as medical and scientific knowledge advances.

For example, Cannabis compounds are known to have beneficial effects on medical conditions such as chronic pain, paralysis, neuropathy, Crohn's Disease, inflammatory bowel disorders (IBS and IBD), glaucoma, PTSD, anxiety, seizures, post-traumatic stress disorder, opiate dependency, epilepsy, autoimmune disorders, autism, tumors, cancer, and nausea. Data is accumulating relating to the effects of Cannabis on these conditions. This data relates to, for example, individual user characteristics, states of the condition, and user responses. The systems, methods, and apparatus as described herein, in various embodiments, enable a user who has a condition in which data is available to benefit from prior knowledge, and conveniently formulate a beneficial formulation tailored to this user's needs based on the user characteristics, their physical states, and initial responses. Then, as the data improve and the user changes, the formulation can, in accordance with various embodiments described herein, evolve too, to fulfil a user's changing needs.

The problem as solved by various embodiments described herein, starts with the complexity of the Cannabis plant and the hundreds of compounds that can be utilized to provide beneficial effects to users. Users have differing physiology and needs, and these are always changing. The systems, methods, and apparatus, described herein, correlates scientific and medical data with a user's personal information, including their specific goals and needs, to achieve formulations tailored to that user's specific goals and needs. Once a dosage of Cannabis is formulated to be tailored to the user's specific goals and needs, the system provides for the delivery of a dosage form in precisely measured liquid and/or solid forms of active compounds, such as cannabinoids, terpenes, and desired adjuvants in doses and formulations tailored to that individual user's goals and needs. Liquid and/or solid forms of Cannabis compounds are used in various embodiments described herein because these forms enable formulations to be prepared with precisely measured amounts of formulation compounds.

Achieving accuracy of delivery is an important problem solved by the methods, systems, and apparatus described herein because users have different sensitivities to Cannabis compounds and Cannabis compounds are known in some cases to produce subtle and countervailing effects. There is a substantial need fulfilled by the invention provided herein in various embodiments because before the inventor's disclosure, users were not able to have Cannabis formulations tailored to their own needs and delivered in accurate and consistent forms. The methods, systems, and apparatus described herein, enable a wide range of opportunities for obtaining beneficial effects derived from Cannabis compounds that were previously lost and can meet the needs of users whether they are generally therapeutic, wellness based, specific disease related, specific disease therapeutic, psychologically enhancing, pain reducing, recreational, and combinations thereof.

While the systems, methods, apparatus described herein can be used for any bioactive compound, they are especially well suited to tailoring and delivering formulations of Cannabis compounds. Due to the changing needs of users and their evolving sensitivity to Cannabis compounds over time, the wide variety of useful bioactive Cannabis compounds, and the growing Cannabis databases accessible to users, there is a strong need for the systems, methods, and apparatus, described herein in various embodiments that can tie together these changing variables to achieve tailored Cannabis formulations for individual users. In various embodiments disclosed herein, the systems, methods, and apparatus dispense and formulate tailored doses of Cannabis compounds through the use of the wellness APP that utilizes subject data, public databases, and artificial intelligence to achieve tailored Cannabis formulations for individual user needs and goals.

Provided herein, in some embodiments is a platform comprising the interactive wellness App, public and/or private databases featuring research related to Cannabis and human health, Cannabis, public policy, Cannabis related research, Cannabis compounds, or combinations thereof, for providing Cannabis dosing formulations, tailored to individual users, and a dispenser to conveniently provide the tailored Cannabis dosing formulations. In various embodiments the tailored dosing formulations can be prepared in accordance with the dosing formulation determined by the wellness App outside the home in, for example, a laboratory, or prepared in the dispenser where the dispenser is configured to prepare Cannabis dosing formulations. Where the Cannabis dosing formulation is prepared in a laboratory, solution and solid forms of the compounds of the dosing formulation can, in some embodiments, be delivered to the home and dispensed from cartridges, pills, solid forms, or combinations thereof. Dosing formulations are constructed such that a user will be able to dose precise amounts of the Cannabis compounds, as determined by the tailored Cannabis dosing formulation.

In various embodiments, Cannabis compound formulations can be stored in a safety prepackaged cartridge. In some embodiments, prepackaged cartridges can dispense either a liquid formulation or solid formulation according to the amount of dosing. A solid formulation can in some embodiments be in the form of pills, gel tablets, capsules, or combinations thereof. In some embodiments, solid formulations can be in a form that can be solubilized by the user.

In some embodiments, once the wellness APP determines a dosage, it communicates this securely with a dispenser. The dispenser then verifies and authenticates the user with password, ID, or other authentication methods. Once authenticated, the THC liquid dispensed could be, for example, to provide 10 milligrams (mg) of THC, while adding a 20 mg CBD pill, to combine with the 10 mg of THC to provide a dosing formulation of 10 mg THC and 20 mg CBD. In some embodiment, the liquid portion of the formulation can be dispensed from a liquid channel spout into a cup underneath and the solid pills are dispensed on a solid holding chamber, separate from the liquid channel spout.

The wellness App, or platform, as described herein in various embodiment, retrieves information from databases, and can, in some embodiments, be designed to automatically update, to utilize available features to encompass macro trends and impact scores of the various journal articles, and enhance incorporated algorithms by including impact scores of journal articles cross referenced with topics and sub topics, tracking of articles by topic and sub topic by day/month/year, and an alert system whereby macro trends in data publishing can be cross referenced with similar trends in the data our platform is collecting.

In some embodiments, provided herein is an apparatus for providing user tailored Cannabis formulations, comprising a wellness App on a communication device, for gathering user data, interfaced with an artificial intelligence (AI) system configured to cross reference one or more database of Cannabis compounds, herbal compounds, or combinations thereof, and a dispenser comprising a housing structure which comprises a water tank and a mixing cell, one or more cartridges for storing and mechanically distributing measured amounts of compound to the mixing cell in operable connection with the wellness App (software application), and a dispensing operating intelligent controller operably connected to the wellness App, and a dispensing mechanism fluidly connected to the mixing cell and under the control of the dispensing operating intelligent controller.

In some embodiments, the communication device is selected from the group consisting of smartphone, tablet, mobile internet device, laptop, wearable computer, computer, personal digital assistant and combinations thereof. In some embodiments, the AI system processor comprises a pharmaceutical dosage formula algorithm and is capable of evolving treatment and dosages to be tailored to a user.

In some embodiments, the database is selected from the group consisting CDRmed, UCLA Health, PubMed, The United Medical Language Systems (R) (UMLS), Metathesaurus (R), WebMD, Cannabis med, National Center for Biotechnology Information (NCBI), Clinical Trials Gov, Annals of Internal Medicine CannaMD, Americans for Safe Access, BMJ.org, American Cancer Society, Cannabis Community Care & Research, Eureka Alert, Medicinal Cannabis, Animal Health Studies Database and combinations thereof.

In some embodiments, provided herein is a system for tailoring Cannabis dosage formulations to a user comprising a wellness App which comprises an artificial intelligence connected software application for iteratively establishing formulations based on a user entered personal data and public database of Cannabis compounds, and a dispenser, for dispensing tailored Cannabis dosage formulations.

In some embodiments, the wellness App is a smartphone APP. In some embodiments, the wellness App is in communication with the apparatus dispenser using a communication interface. In some embodiments, the apparatus dispenser can mix. In some embodiments, the communication interface is selected from NFC, WIFI or Bluetooth. In some embodiments, user entered personal data is stored on a computer readable medium.

In some embodiments, provided herein is a method for obtaining tailored Cannabis dosing formulations for a user, comprising collecting user information by having the user engage with and enter information into an interactive wellness APP, accessing Cannabis compound databases, establishing a baseline Cannabis dosage by correlating the Cannabis database to fit with the user information, preparing a baseline Cannabis dosage comprising two or more Cannabis compounds in amounts that achieve the baseline Cannabis dosage, observing user responses to the baseline Cannabis dosage to provide user response data, entering the user response data into the wellness APP, wherein the wellness App determines a tailored Cannabis dose for the user wellbeing.

In some embodiments, user information is selected from medical conditions, symptoms, pain levels, and prescription medications. In some embodiments, the method further comprises optimizing dosage treatment by entering user data after dosing with the tailored Cannabis dose and syncing the data with an artificial intelligence algorithm for optimizing the Cannabis dosing with data from Cannabis databases in real-time.

In some embodiments, optimizing dosage treatment comprises analyzing data relating to Cannabis dosage and human wellbeing and comparing with user information to achieve an optimized Cannabis formulation. In some embodiments, human wellbeing is physical wellbeing. In some embodiments, human wellbeing is psychological wellbeing.

In some embodiments, the method further comprises dispensing tailored Cannabis dosing formulation to the user. In some embodiments, the method uses a dispenser described in various embodiments herein. In some embodiments, the Cannabis compounds are selected from CBG, CGC, CBD, THC, CBGa, CBCa, CBDa, THCa, and combinations thereof. In some embodiments, the Cannabis compounds are selected from CBG, CGC, CBD, THC, CBGa, CBCa, CBDa, THCa, terpenes, and combinations thereof.

In some embodiments, once a user agrees and accepts via the wellness App, the dispensing of liquid and solid Cannabis, herbal, and and/or pharmaceutical compounds custom tailored to a user's needs can be dispensed. In some embodiments, this can be a one time serving for the user and future dosing can be tailored according to changing user needs an goals. A regimen of dosages tailored to be suitable for individual users in accordance with various embodiments described herein, provides the greatest benefits achievable from Cannabis with respect to efficacy, wellness, and entourage effects.

### BRIEF DESCRIPTION OF THE DRAWINGS

FIG. 1A shows a back view of a dispenser for dispensing tailored dosage formulations;

FIG. 1B shows a side view of a dispenser for dispensing tailored dosage formulations;

FIG. 1C shows a front view of dispenser for dispensing tailored dosage formulations;

FIG. 1D. shows a top view of a dispenser for dispensing tailored dosage formulations;

FIG. 1E. shows a detail of the dispenser illustrating the solid dispensing region;

FIG. 1F. shows a detail of the dispenser illustrating a liquid dispersing structure, and

FIG. 2 shows a flow chart of the components and operating flow of Application Software, AI database, and communication platform and dispenser in accordance with some embodiments described herein.

### DETAILED DESCRIPTION

The inventor provides a solution to the complexities, inaccuracies, and inconsistencies that hinder Cannabis dosing by providing, in various embodiments, methods, systems, and apparatus combining a wellness App that incorporates artificial intelligence with a dispensing apparatus and access to evolving public information, i.e., data, on Cannabis use and effects. The various embodiments described herein also include the methods, systems, and apparatus, by utilizing other scientific and pharmaceutical databases, to improve Cannabis formulations by utilizing other pharmaceuticals and/or Cannabis adjuvants.

As used herein, the term “wellness App” refers to a mobile application program that offers health-related services on communication devices, such as smartphones, tablets and other communication devices. Where the wellness App is on a smartphone, it is, in some embodiments herein, referred to as a Smartphone App. In the context of this disclosure, the smartphone App is the same as the wellness App. The wellness App (Also referred to herein as “App” or “Application” of “software”) tracks user wellbeing by tracking fitness activity, diet with nutritional guidance, stress reduction, meditation, community building, social network and sleep analysis. The wellness App interactively collects user information, interfaces with databases, and through an incorporated artificial intelligence algorithm, formulates tailored Cannabis dosage formulations in accordance with the needs of individual users.

As used herein, “tailored dosing of Cannabis formulations” refers to dosing with a Cannabis formulation in such that accurate amounts of Cannabis compounds, adjuvants, and other compounds or herbs are provided to a user in accordance with a dosing determination based on information provided by the user and interfacing with available databases. As used herein, in various embodiments, “tailored dosing formulations” can be two or more Cannabis compounds in determined amounts for each compound in accordance with user needs as determined through methods described herein in some embodiments. Tailored dosing formulations can, in some embodiments, comprise in addition to the two or more Cannabis compounds, additional compounds, herbs, and adjuvants where desired by the user.

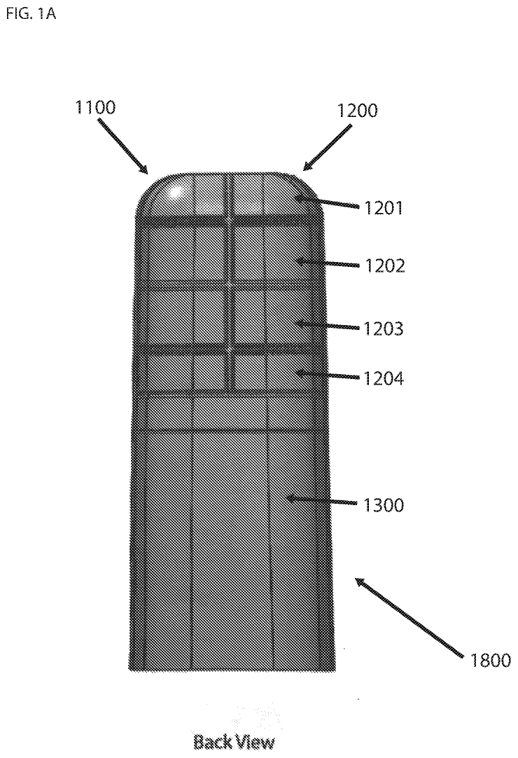

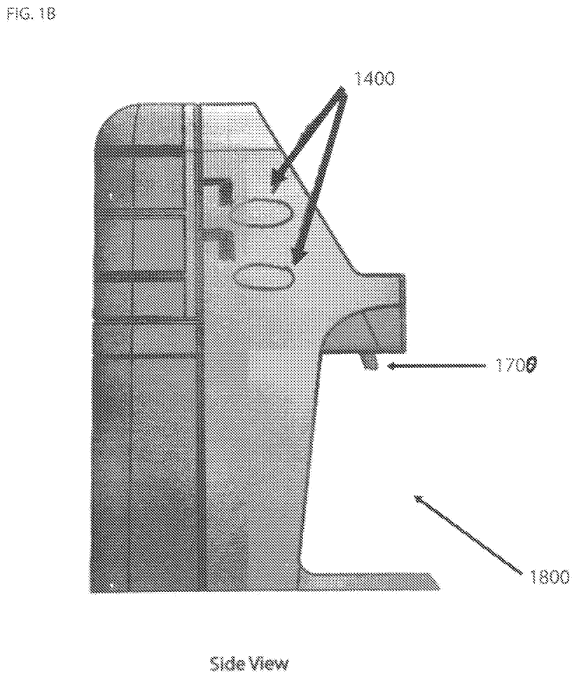

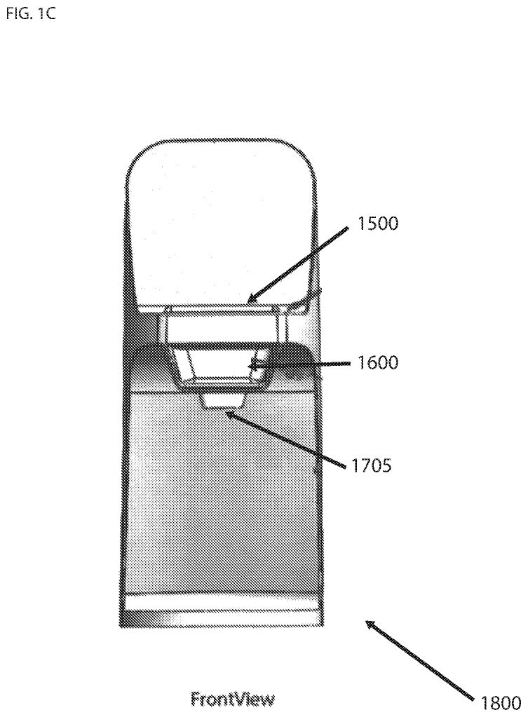

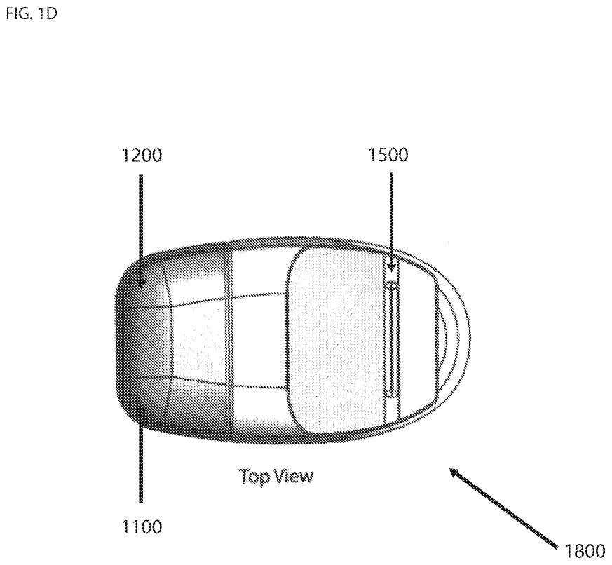

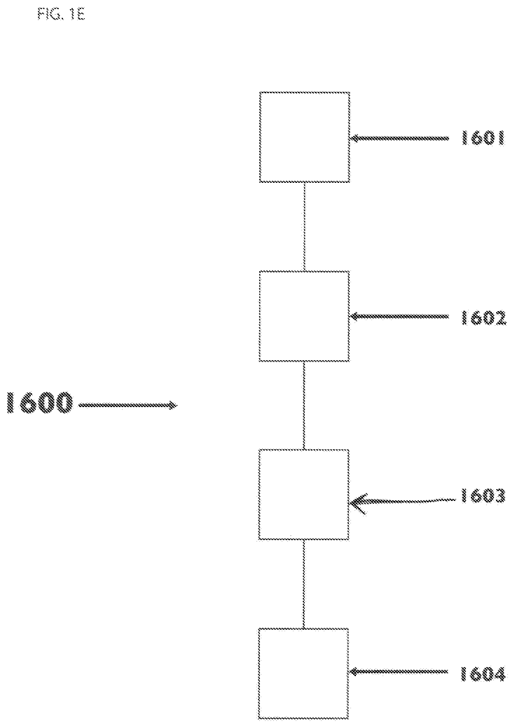

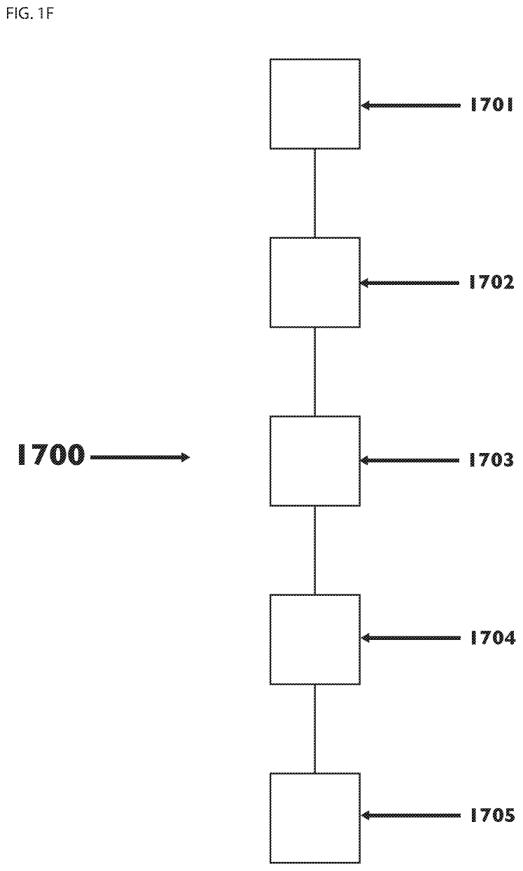

As used herein, dispenser “apparatus for dispensing”, also referred as a “dispenser”, “dispenser apparatus”, “dispenser machine”, “hardware”, or “serving apparatus,” is the apparatus from which a user can obtain, in some embodiments, tailored dosing of Cannabis formulations, that have been tailored to the user in accordance with various embodiments described herein. As used herein, the terms apparatus for dispensing, dispenser, dispenser apparatus, dispenser machine, hardware, and serving apparatus are used interchangeably. The dispenser is illustrated, in some embodiments, in FIG. 1A, FIG. 1B, FIG. 1C, FIG. 1D, and FIG. 1E.

Dispensing mechanism, in some embodiments, refers to an intelligent dispensing operating controller that takes instructions from the wellness App and which comprises compartments, pumps, precise mixing means, precise measurement means, and precise sensors for dosage formulation and dispensing.

The combination of the wellness App, dispenser, and available Cannabis compounds with associated data, enables a user, in various embodiments described herein, to continually improve their Cannabis dosing and concomitant health and wellbeing. The inventor recognizes that the systems, methods, and apparatus, as described herein, will enable users to continually improve their Cannabis dosing as additional scientific knowledge and Cannabis data become available.

In various embodiments, the systems, methods, devices, and apparatus described herein provide personalized, or tailored, treatment or therapies for individual users. The wellness App, in various embodiments, can gather data from a vast pool of users of Cannabis, and herb, for wellness and cure, and store, compare, analyze to fit data compiled from users with similar backgrounds, sex, age, pains, diagnosis, diseases and similar symptoms. Using the wellness APP, a user can, for example, find and select a peer-to-peer and crowd-sourced matches, and then, in some embodiments, can select a recommended baseline treatment plan from the wellness App. In some embodiments, user data will be kept secure using cybersecurity data systems known to persons of skill in the art.

The wellness App, in some embodiments, can communicate with the apparatus for dispensing user tailored Cannabis formulations via wifi or bluetooth to communicate the tailored dosing formulation from the available full spectrum cannabinoid, such as THC, CBD, and other minor Cannabinoids and Terpenes, to provide for an optimal personal dosage formulation provided directly to the user.

In contradistinction to other ways of ingesting, or absorbing, Cannabis compounds, the pre-measured and exact CBD:THC dosage cannot otherwise be obtain, such as by smoking the flower of the Cannabis plant. The dispenser in some embodiments can have multiple slots for tank inserts for powder or liquid or solid pills holding tanks. In some embodiments, the water tank will serve as an agent for solubilizing the herb powder and/or pharmaceutical grade medicine. In some embodiments, a tank that holds flavors (orange, lime, etc.) can be provided. In some embodiments, certain tanks can hold pre-made CBD formulations in measured (such as 1 mg) doses, and other tanks can hold pre-made THC formulations in measured (such as 1 mg) doses, and these formulations, where desired can be combined in determined ratios to provide for proper ratio dosage combinations in milligram doses for proper ratio dosage combinations and while other tanks hold.

As used herein, the terms “user”, “person”, and “subject” are synonymous refer to a human being.

Dosage formulation, dosing, tailored dosing of Cannabis formulations, formulations, as used herein refer to combinations of two or more Cannabis compounds with defined amounts of the Cannabis compounds to be in doses. The dosage formulations can, in some embodiments, comprise adjuvants, herbs, other pharmaceuticals, or combinations thereof. The characteristic of tailored dosing formulations that is provided in various embodiments herein, is that the amounts of the Cannabis compounds in the dosing formulation have been determined to fulfil the requirements, desires, goals, or needs of the user. That is that they have been personalized, or tailored, to deliver what would be most beneficial to the user.

Cannabis dosing can be complex and specific treatment knowledge and nuance on dosage formulations is generally not available and difficult to predict a priori. and optimal dosages of CBD vary from person to person. An effective dosage can range from as little as a few milligrams of CBD-enriched Cannabis oil to a gram or more. In various embodiments, users can establish their optimal dosage profile by starting with small doses of high CBD/low THC. Initial dose ranges can be varied depending upon the user's prior experience with Cannabis, body mass, and other considerations relating to their physical and psychological conditions. General recommendations can include, in some embodiments, taking a few small doses over the course of a day rather than one big dose, using the same dose and ratio for several days, and/or observing the effects and if necessary adjust the ratio or amount. Cannabis compounds have bi-phasic properties, which means that low and high doses of the same substance can produce opposite effects.

Therefore, the assessment of various conditions and variables to achieve optimal dosing and/or formulations of Cannabis is not routine for doctors, individual users, or lab technicians lacking knowledge and the protocols to ascertain the necessary steps one needs to take into consideration to develop tailored Cannabis formulations from Cannabis or even the individual major Cannabis compounds CBD and THC. Doctors, users, lab technicians simply cannot calculate all the variables for achieving optimal Cannabis formulations on their own. For example, small doses of Cannabis tend to stimulate; large doses sedate. Too much THC, while not lethal, can amplify anxiety and mood disorders. CBD has no known adverse side effects, but an excessive amount of CBD could be less effective therapeutically than a moderate dose. Less is more is often the case with respect to Cannabis therapy. These considerations reflect the subtleties in dosing and formulation tailoring and/or development and highlight the need for the systems, methods, apparatus, and or devices provided by the current disclosure.

In various embodiments described herein, the hardware and software platform provides for these determinations of tailored Cannabis formulations for precise dosing combining physical and psychological attributes of the user, while assessing, calculating, matching the user attributes with data from accessed databases to obtain a treatment plan with a daily recommended tailored

Cannabis compounds, in various embodiments, include cannabinoids and terpenes. Formulations can also comprise adjuvant compounds. Various bioactive compounds can, in some embodiments, be included in formulations provided by various embodiments described herein, and the methods, systems, and apparatus described herein enable the tailoring of these formulations to individual user needs.

Tailored formulations, or dosage forms, as used herein in various embodiments, include Cannabis compounds and are deliverable to users such that determined, or measured, amounts of each Cannabis compound can be delivered to provide a dose consonant with the user's needs as determined by various embodiments described herein.

Cannabis compounds include, for example, cannabinoids such as CBG, CGC, CBD, THC and minor Cannabinoid acids CBGa, CBCa, CBDa, THCa and Terpenes, and the like. In some embodiments, the bioactive ingredients are chemical derivatives of cannabinoids such as CBG, CGC, CBD, THC and minor Cannabinoid acids CBGa, CBCa, CBDa, THCa and Terpenes.

Cannabis compounds can be isolated in some embodiments in accordance with methods understood by persons skilled in the art. In some embodiments, the Cannabis compounds, and other bioactive ingredients, can be produced in a micro-organism using the methods of synthetic biology. In some embodiments, compounds are prepared in accordance with methods of synthetic organic chemistry. Compounds used in the tailored formulations can, in some embodiments, be purchased from vendors.

There are many Cannabis compounds. A recent publication indicates that there are more than 60 cannabinoids and over 400 chemical compounds in the Cannabis plant. see Atakan, Ther Adv Psychopharmacol, 2(6) 241-254 (2012). As Cannabis compounds become better known, additional cannabinoids, and other Cannabis, compounds will find use in achieving human wellbeing. The systems, methods, and apparatus described herein, in various embodiments, can achieve tailored formulations that include any Cannabis compound, from those well characterized to those as yet unknown, once data becomes available, either from a user directly, or from databases as described herein.

In some embodiments, the hardware and software platform provided herein can provide personalized Cannabis medicinal formulation dosages that are specifically tailored to individuals. In some embodiments, the formulations can be used for treatment regimens. In some embodiments the treatment regimens can be tailored to individuals.

An example of the nuanced bespoke formulations that can be achieved by the tailored dosing of Cannabis formulations provided, in some embodiments, by the systems, methods, and apparatus provided herein is that depending on user goal, combinations of Cannabis compounds, and tailored formulations, can be prepared that produce psychoactive and PAFR (psycho-active fluid responses). PAFR, as used herein, refer to a euphoric psycho activity without common side effects, such as dry mouth, red eyes, paranoia, sleepiness that are sometimes associated with Cannabis consumption. In various embodiments, a refined quality and experience can be achieved through tailored formulations in accordance with individual user needs. This experiential variance of mental and physical state which the newly discovered individualized Cannabis dosage achieves comes from the formulation platform described herein. The formulation platform described herein, in various embodiments, expands on intended traits and thus controls the effects and desired states of psycho & physiological activity and thus provides qualities and experiences tailored to individual needs and desires. This experiential variance of mental and physical states which the Cannabis dosage and formulation platform described herein can achieve enables the expansion and discovery of dosages and formulations to achieve desired traits and effects previously not recognized. Thus, the approach taken here also provides a pharmaceutical formulation discovery platform. The invention, in various embodiments, comprises, systems, methods, devices, and apparatus including software, apps, hardware, and combinations thereof.

Table 1 shows the major Cannabinoids found in various proportions in any given Cannabis plant and illustrates the major compounds and what they are helpful for.

TABLE 1
Cannabis Plant Compounds

| Cannabinoids | CBD/CBDA | CBG/CGA | MC/ CBCA | THC/THCA/THCV |
|---|---|---|---|---|
| Benefits/Treatment | Relieves Anxiety | Insomnia | Inhibits Cancer cell growth | Inhibits Cancer cell growth |
| Benefits/Treatment | Relieves Pain | Inhibits Cancer cell growth | Promotes Bone Growth | Suppresses Muscle Spasms |
| Benefits/Treatment | Reduces Seizures | Promidtes Bone Growth | Reduces Inflamation | Stimulates Appetite |
| Benefits/Treatment | Reduces Blood sugar levels | Slows Baterial Growth | Relieves Pain | Reduce Vomiting |
| Benefits/Treatment | Inhibits Cancer cell growth | Reduces Inflamation | Treats Fungal Infection | Reduce Nausea |
| Benefits/Treatment | Redoction of Inflamation | Relieves Pain | | Relieves Pain |
| Benefits/Treatment | Mood Disorders | | | Aids Sleep |
| Benefits/Treatment | Reduces risk of artery blockage | | | Suppresses Muscle Spasms |
| Benefits/Treatment | Relieves Asthma | | | Reduces Convulsions |
| Benefits/Treatment | Diabetes | | | Reduces Seizures |
| Benefits/Treatment | Vasorelaxant | | | |
| Benefits/Treatment | PTSD | | | |
| Benefits/Treatment | OCD | | | |
| Benefits/Treatment | Antibacterial | | | |
| Benefits/Treatment | Neuro-Protective | | | |
| Benefits/Treatment | Promotes Bone Growth | | | |

Cannabis therapeutics, as understood herein, are personalized medicines. There is no single ratio or strain or product that's right for everyone. In various embodiments, the wellness Apps, platform, devices, systems, and apparatus described herein enable users to optimize therapeutic use of Cannabis by finding the proper combination of CBD, THC, and or other compounds that provides for the best outcomes for the user. What works best for a person will be a combination of physiological, personal, psychological, and combinations thereof, factors that work best for each individual. For example, a person's sensitivity to THC can be a key factor in determining the appropriate ratio and dosage of a CBD-rich medicine.

Highlighting the need for personalized options in Cannabis dosing is that there is a great variation in response to Cannabis compounds and interests in users in the effects of the Cannabis compounds. Many people enjoy the Cannabis high and can consume reasonable amounts of any Cannabis product without feeling too high or dysphoric. Others find THC unpleasant. CBD can lessen or neutralize the intoxicating effects of THC. Therefore, for example, a greater ratio of CBD to THC means less of a high. In some states with medical marijuana laws, Cannabis oil concentrates and other products with varying ratios of CBD:THC are available so users can adjust or minimize psychoactive effects to suit their needs and sensitivities. Those who don't like THC have the option of using various dosage forms without the high by using a CBD-rich remedy with only a small amount of THC. But a low THC remedy, while not intoxicating, is not always the most effective treatment option. The apps, platform, devices, systems, and apparatus described herein, either alone or in combination, enable the user to administer consistent, measurable doses of a CBD-rich remedy that includes as much THC as a person is desires, needed or is comfortable with.

In some embodiment various CBD:THC ratios can be prepared for different conditions. As research continues, additional data will become available. The systems and methods described herein can utilize additional information as it becomes available by integrating new data, recognizing patterns as they begin to emerge, and utilizing the intelligence of the AI Algorithm to create improved dosage formulations.

Dosing options vary depending on the condition or needs of the user. For example, anxiety, depression, spasms, psychosis, and seizure disorders, generally are found by users to respond well by starting with a small dose of a CBD-rich remedy with little THC. For cancer, autism, and many other diseases, a balanced ratio of CBD and THC has been found to be beneficial. Extensive clinical trials conducted outside the United States have shown that a 1:1 CBD:THC ratio can be effective for neuropathic pain. What's more, while these finding can be used as starting points in dosage formula development, each user will be different based on all the factors that make us unique changing individuals.

Some people use Cannabis products with different CBD:THC ratios at different times of the day (more THC for sunlight hours, more CBD at night). Almost any Cannabis strain or product theoretically could benefit a wide range of autoimmune and inflammatory disorders because THC and other Cannabis components activate the CB2 cannabinoid receptor, which regulates immune function.

Provided herein, in some embodiments, is a platform comprised of hardware and software designed to determine and prepare, and/or create Cannabis compound formulations tailored to a user's individual needs by combining chemical components from Cannabis plants, and where desired other bioactive agents and/or adjuvants.

In some embodiments, the systems, methods, and apparatus described herein use an artificial intelligence interfaced software system incorporated within a wellness App such that a person's current and evolving needs can be met by experiential use and feedback of user entered data in coordination with available Cannabis, and other health related, databases. In some embodiments, the available databases are in the IoT (internet of Things) via the cloud and/or Database. In some embodiments, the systems, methods, and apparatus described herein are provide iterative intelligence based formulating of all compounds that would achieve benefits for a user.

In some embodiments, the wellness App, as described herein, is used as a smartphone application/software that provides a peer-to-peer, crowd-sourced Cannabis wellness information and treatment recommendation social platform which serve individual dosage formulation via the dispensary platform. In some embodiments, the smartphone application/software links user possible HIPAA (Health Insurance Portability and Accountability Act) grade data safety to hardware described herein. In some embodiments, the wellness App is a smartphone application/software but the wellness App in various embodiments can be associated with any computing device. In some embodiments the wellness App is a smartphone App. In some embodiments, the wellness App is a tablet App, mobile internet device App, laptop App, wearable computer App, computer App, personal digital assistant App or combinations thereof.

In some embodiments described herein, an App, Application software, referred herein as a wellness App, is used in the methods, systems, an apparatus. The wellness App gathers user data, uses AI (Artificial Intelligence) to collect data, cross reference other databases of relevant user information, types of illnesses, types of treatment information, dosage information. The Wellness App collects user data such as age, sex, weight, illnesses, a physical profile, and a psychological profile. The Data, in some embodiments, is then paired with reasoning, learning and other general intelligence as well as sources of information, such as peer-to-peer knowledge, and the treatments, crossed referenced with workable, proven dosages, and their success rate.

The AI algorithm enables the dosage formulation engine provided by the various embodiments described herein. The AI can, in various embodiments, calculate the best possible successful path for treatment and recommends dosages and/or formulations to the user.

In various embodiments the wellness App (APP) provides secure, simple, and intuitive new user boarding and health data retrieval and data storage. In some embodiments, the wellness App is developed such that data storage is HIPPA compliant data storage. In practice, in various embodiments described herein, the wellness App asks new users to input their information, such as medical conditions, symptoms, pain levels, prescription medications, and other features and then suggests an initial dose formulation. After the user ingests the initial dose formulation, the wellness App then prompts users to rate the effectiveness of the treatment with respect to various parameters such as pain level incorporating this new information. The wellness App provides for accurate reading and serving of metered dosing of liquid and solid Cannabis extracts that can include, in various embodiments, cannabinoids, terpenes, and/or adjuvants, in accordance with the user's needs.

In some embodiments, the wellness App is designed to store data in anonymous and encrypted form while tracking consumption and dosage effectiveness. The wellness App, in various embodiments, therefore enables the user data obtained through experience to be anonymously shared with doctors, caregivers, other users, and combinations thereof. The data shared with other users can be in the form of contributions to established databases.

The wellness App can, in various embodiments, track a user's anxiety, inflammation, pain and well-being. It will record the user's Cannabis intake and user feedback. The wellness App will learn to modify dosages based on dosing and responses and will factor in genetics, metabolism, tolerance as well as other types of user-generated data such as height, weight, age, diet, physical activity, and combinations thereof.

In some embodiments described herein, the wellness App is a phone App that enables Peer-to-peer sharing of experiences and illness diagnoses from treatment accounts and descriptions such as Project CBD, a non-profit educational service and in abstract clinical neuroscience publications such as National Center for Biotechnology Information, American Cancer Society, Leafly, Scientific Published Endocannabinoid System, where developments and data relevant to Cannabis formulations is collected. The wellness App, in some embodiments, will mine data from a variety of sources, such as, for example, CDRmed, UCLA Health, PubMed, The United Medical Language Systems (R) (UMLS), Metathesaurus (R), WebMD, Cannabis med, National Center for Biotechnology Information (NCBI), Clinical Trials Gov, Annals of Internal Medicine CannaMD, Americans for Safe Access, BMJ.org, American Cancer Society, Cannabis Community Care & Research, Eureka Alert, Medicinal Cannabis, Animal Health Studies Database, Peer-Reviewed Studies on Medical Marijuana, Clinical studies from Institutions such as Israel Medical Marijuana Institute, clinical studies, group studies, writings for using herbal and Cannabis therapy, and combinations thereof. The wellness App, in some embodiments, use AI to gather knowledge from around the world. The wellness App can, in various embodiments, correlate of data from numerous sources while also parsing extensive knowledge from stored medical definitions, relationships and studies such as Pubmed, The United Medical Language Systems (R) UMLS), Metathesaurus (R), WebMD, Cannabis-Med.org, National Center for Biotechnology Information (NCBI), Clinicaltrials.gov, Annals of Internal Medicine, healer.com, and the like.

Persons of skill in the art understand that the databases and sources for information listed herein are only examples and that as new or additional databases, journals, groups, etc. become available, they can be accessed and used in accordance with various embodiments described herein.

In some embodiments, the Artificial Intelligence layer interacts to capture data by voice inputs (using similar methodology as Alexa from Amazon or Siri from Apple) and/or by entering the answers by typing. The wellness App can aggregate data from other health care, patient treatment, studies, research labs and cross-reference databases available from, for example, therapy centers, researches in the medical Cannabis space, and users with similar problems, symptoms and illness and their treatments. Based on that information, the AI can provide recommendations based on previously successful formulation and usage regimens. Once the user chooses a formulation dosage of herbal, Cannabis and/or pharmaceutical compounds combination in accordance with one or more recommendation, the dispensary hardware will dispense a formulation (dosage) of herbal, Cannabis and/or pharmaceutical compounds via wireless connected, user authentication and/or paired IoT (Internet of Things) connection protocol for dispensing hardware.

The wellness App can, is some embodiments, use various AI (Artificial Intelligence) techniques to act as intelligent sourcing agents, it can understand speech, can record and takes into account, for example, a user's age, body weight, sex, symptoms, severity of pain and many other physical ailment conditions, sleep patterns, appetite levels, mood and physical strength, as some examples.

In some embodiments, the wellness APP interfaces with a dispenser apparatus to intelligently and automatically serve blended tailored dose to a user while tracking the product levels so that the user will not run out. In some embodiments, when the dispenser apparatus detects low-product levels, it can automatically order replacement Cannabis blends in liquid or solid, lab-tested, adult-use-only pre-packaged cartridges if the user has requested such services.

In various embodiments, the wellness App communicates with databases, an AI platform and a dispenser apparatus (also referred to as a “serving apparatus”, or “dispenser”). In some embodiments, the wellness App, AI platform, and dispenser apparatus provide an interactive system for continuously tailoring of doses to be consonant with a user's current and changing needs. Because the interactive system, in some embodiments, is continuously adjusting for database and user changes, the interactive system enables the user to receive continuously improving dosing formulations to meet their needs and goals for health and wellbeing. In some embodiments, both physical and psychological parameters of interest to the user can be included in formulation development. For example, in various embodiment, each interaction session of use, each serving session, each metered dosing of liquid and/or solid Cannabis extracts, compounds, all of these interactions can be recorded to building the user's profile depth, efficacy results of results, and the user's overall wellness and these parameters can be correlated with the metered dosing to enable the user to continue to improve the responses and improve the Cannabis dosing formulation.

By constantly gathering information, in various embodiments, the platform's AI engine can continue to create dosing best suited to a user's needs as they evolve. In various embodiments, by tracking each session, the AI algorithm learns, adjusts and in conjunction with the dispenser and wellness App, achieves and serves optimal dosing for a user to feel better and for longer durations without over-medicating or under-medicating.

The wellness APP can be designed and programed by using JAVA or Xcode graphical interface. There are numerous agencies and app development platforms to use and designing a wellness App in accordance with embodiments described herein is understood by persons of skill in the art. For example, App builder/creator, TheAppBuilder, and App Pie can be used in some embodiments. The UX and UI can be refined, in some embodiments, to maximize a user's ease of interface. Additional elements on the App can include, for example, swipe, a touch screen, and typed into.

In some embodiments, voice activated inputs can be used with Questions and Answers for the user. Example, the APP may ask, “please tell me your pain level now? 1 to 10, 1 being little pain or 9 being extremely painful.”

The wellness App, in accordance with various embodiments, works on, for example, such parameters as understanding a user's reasons for using Cannabis, will assess how much pain or discomfort or severity of illness the user has, will work towards capturing the user's experiences, tolerance, speed of onset, duration and satisfaction, and determine was the comparable level of pain before and after Cannabis dosing.

In some embodiments, the wellness App described herein can use AI speech recognition, chat-bots will problem solve to maximize the chances of success towards a goal for best treatment path and best dosage formulation per serving. It will gather all the information from user such as their personal and medical profile, their sleeping and exercise profile, their overall state of wellness, their physical pain and discomfort levels, their mental state, their daily improvements, if any. In some embodiments, data can be stored in the cloud and can be synced with the individual's user database. In some embodiments, data can be organized and mined with big clinical studies, case report datasets with machine learning for symptom treatment correlations. In various embodiments, data can be automatically compared, matched and given qualitative analysis with dosage formulation recommendations for treatment to relief user's symptoms, illnesses, pains, mental stress, and related conditions.

Before and after each use, the wellness App, in some embodiments, can use Chatbot or other Artificial Intelligence add-ons to engage users to provide feedback and rate their sensations, overall state of the body, mental state, and physical wellbeing. This information can be gathered over time as the user interacts with the wellness App in connection with the dispenser. The wellness App can adjust real-time based on the information gathered and formulate a unique combination and dosage (formulation) tailored for the user's next dose.

The AI, in some embodiments described herein can search, compare, optimize data to provide users with the most intelligent, suitable combination based on the desired medical effect recognized from the information shared by others from their successful treatments. The start of therapeutic dosage will be dependent on many factors such as age, weight, severity of illness, types of illness. The dosage will vary daily to accommodate these factors.

In some embodiments, the wellness App can synch with other devices such as wearable IoT devices. For example, Apple Watch or Fitbit for vital statistics, data sets like heart rate, metabolic levels, exercise routines and exertion indications can be used in conjunction with the wellness App in some embodiments. This additional data provides added insights into the user's physical activities and vital signs to find deeper meanings and correlation for, in some embodiments, additional iterations and dosage formulations.

In some embodiments, described herein is a system, method, and apparatus, that uses a wellness App that gathers user data in conjunction with Artificial Intelligence to gather and process data related to sex, age, weight and symptoms or illnesses. The wellness App can, in some embodiments, also pull from larger data pools of similar users, past scientific research, and successful treatment outcomes for the herbal and Cannabis users. This information is then compiled, and the platform's AI algorithm will use this information to create a personal dosage formula specifically designed for user. Once the user agrees to a recommendation, the wellness App will prompt for next steps, which will then serve the exact herbal, Cannabis and/or pharmaceutical compound, pill or gel dosage for the user. After the dose has been ingested, the user will confirm taking the dispensed amount and the wellness App can automatically update the user's usage database. This user information will be gathered and, in some embodiments, become part of the peer-to-peer, crowd-sourced and crowd-generated content that will continue to build on the efficacy, successful past treatments from aggregate users around the world for their herbal, Cannabis and/or pharmaceutical treatment.

In some embodiments, the dispenser is an IoT Smart Hardware, a wellness, on-demand, personal, private herbal, Cannabis pharmacology smart home appliance. It can sit on any counter, such as a kitchen counter, which after authentication, will dispense liquid and/or solid herbal, Cannabis and/or pharmaceutical compounds and/or pre-made oral pills formulations real-time based on an individual's treatment path.

In some embodiments, the IoT Smart Hardware can receive instructions of the formula and dosage via a secure connection via Wifi or Bluetooth. In some embodiments, the dispenser can dispense a tailored dose accurate to a tenth of a Milligram blend of herbal, Cannabis and/or pharmaceutical compounds concentrate extracts in either liquid or solid output. In some embodiments, the user can receive a final dosage formulation and have the final mixture blended into a liquid and/or pills, ready to be served with the touch of an authenticated password on their smart phone's or other smartphone device.

In some embodiments, the IoT Smart Hardware described herein can take commands from the wellness App (software/application) and can dispense a dosage combining Herbs and Phytocannabinoids (such as CBG, CGC, CBD, THC and minor Cannabinoid acids CBGa, CBCa, CBDa, THCa and terpenes.) to relieve symptoms. In some embodiments, the IoT Smart Hardware can dispense on-demand making each serving the personalized formula from the raw and/or pre-packaged material, like a pill or an oil or a powder and will dispense for the user, each time. The purpose of careful tracking to achieve the optimal personalized dosage of CBD and/or THC ratio relates to the condition a user is treating. The compounds dispensed could be extracted from grown plants, or produced by synthetic biology using microbes engineered to produce the compounds of interest, or synthesized using organic synthesis. Companies such as tlscorp.com and Hyasynthbio.com, use engineered microbes to produce targeted compounds that can be used in addition to plant derived compounds and can be used for providing formulations for each individual's unique needs. The systems, methods, and apparatus described in some embodiments herein provide for disease treatment, therapeutic, recreational, and other uses. The capacity to provide tailored dosing formulations for a wide range of circumstances, users, requirements, and account for evolving user and information availability provided by various embodiments described herein is a valuable contribution to wellness and health maintenance not previously suggested in the art.

FIG. 1A shows a back view of a dispenser 1800 as used in some embodiments described herein. The dispenser is used, in some embodiment described herein, in conjunction with the wellness App, to provide an apparatus for providing user tailored Cannabis formulations. The dispenser 1800 has a liquid formula cartridge holder 1100 and a solid cartridge holder 1200 with places for holding a gel tab cartridge 1201, a pill cartridge 1202, a powder cartridge 1203, and a flavor cartridge 1204. The dispenser 1800 also has a water tank 1300 that holds water for mixing with solid components of tailored formulations, in some embodiments described herein. FIG. 1B shows a side view of the dispenser 1800 and illustrates an intelligent dispensing operating controller 1400 that takes instructions from the wellness App and which comprises compartments, pumps, precise mixing, precise measurement, and precise sensors for dosage formulation and dispensing. The controller 1400 comprises circuitry understood by persons of skill in the art. FIG. 1B also shows the liquid outlet 1700 described in greater detail in FIG. 1F. FIG. 1C shows a front view of the dispenser 1800, with a docking station 1500 for a smartphone (not shown), a smartphone can stand on the docking station 1500 in some embodiments. Also shown FIG. 1C is the solid pill dispenser 1600 and the dispenser spout/outlet 1705. FIG. 1D shows a top view of the dispenser 1800 illustrating the liquid formula cartridge holder 1100 and a solid cartridge holder 1200 locations and the phone docking station 1500. FIG. 1E illustrates details (not shown) of the solid pill dispenser 1600, that can be included in various embodiments described herein that can be designed for use with the dispenser 1800, showing a pre-formed formulation cartridge 1601, a controller unit dispenser 1602, a pill separation and count chamber 1603, and an exit tray 1604. FIG. 1F. illustrates the details (not shown) of a liquid outlet 1700, that can be include in various embodiments described herein, a pre-mixed formulation cartridge 1701, a controller volume liquid 1702, a digital pump 1703, a pressure valve 1704, and a dispenser spout (or outlet) 1705.

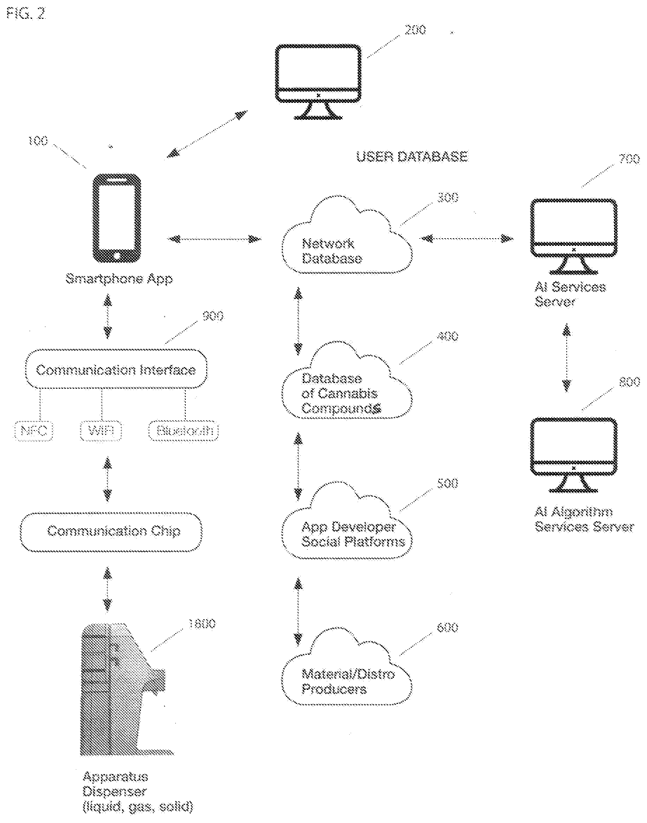

FIG. 2 shows a flow chart of the components and operating flow that can, in some embodiments, comprise a system for tailoring Cannabis dosage formulations for a user. Illustrated are the interactive relationships, for some embodiments, of the Application Software, AI database, and communication platform. In FIG. 2, the wellness App (application operating platform) is a smartphone App 100 and runs on a smartphone that interacts with a user database 200 which captures user data entered in a profile database. A network database 300 connects with cloud based network database of relevant research, findings, peer to peer resources, studies, lab reports and the likes. 400 depicts a cloud based database of herbal, Cannabis and pharmaceutical compounds. 500 are App developer social platforms within the peer-to-peer, social network platform, open sourced network. 600 are linked connections to Material Producers, suppliers and distributors that supply to apparatus. 700 is an AI services provider/server, 800 is an AI Algorithm Services provider/server. 900 is the Communication Interface from the Smartphone device to the Apparatus Dispenser 1800 for liquid, gas and/or solid herbal, Cannabis pharmaceutical compounds. It may connect via NFC, WIFI and/or Bluetooth and the like. 1800 depicts the apparatus dispenser which takes the instructions from the Application Software and dispenses an exact dosage formulation for the user, on demand. Various elements described in FIG. 2 can be combined to provide methods, systems, and apparatus in accordance with some embodiments described herein.

The components, in various embodiments, can be arranged and work together in the following ways. The software used in various embodiments can be a smartphone App that is both on the android and iOS platform. The software will gather, profile, analyze, recommend and authenticate that it is only you who is requesting the medicine. Once the user has selected a final product, the hardware will power up and dispense the proper dosage in a beverage, gel tabs or pill form.

The dispenser (dispensing machine) can, in various embodiments, have a spout for beverage dispensing and there will be gel tab, pill dispense outlet. This machine does not work nor can dispense anything, unless it is paired and authenticated with the user, the app and the dispensing machine.

By blending the various cannabinoid extracts to form the individual dosage to tailor to individual physiological body type, The AI with its unique compiled algorithm can blend extracts and compounds or pills to achieve a ratio of cannabinoid compounds which optimize therapy for a user's condition(s) or goals. The dispenser's backside, in some embodiments, holds tanks for water, flavor, pre-made pills, gels, tablets or oils, powder that contains cannabinoids such as CBG, CGC, CBD, THC and minor Cannabinoid acids CBGa, CBCa, CBDa, THCa and Terpenes. The dispenser can, in some embodiment, detect when it is low on the raw or pre-made materials, it will alert a designated supply chain partner, such as a dispensary or an authorized medical outlet, pharmacy.

In some embodiment, the dispenser can pulls each measured serving and combine the compounds for mixing in a mixer bowl in the dispensing unit. Water can then be added, in various embodiments, to prepare liquid formulations. In some embodiments, he dosage calls for pills to be dispensed, each pill is counted and dispensed according to specifications provided for the formulation and can then be dispensed by the dispenser (hardware).

In some embodiments, tailored formulations are determined for a user using the wellness APP, the formulations are prepared, and then sent to the user for dispensing using the dispenser. The tailored formulations can be prepared, in accordance with methods known in the art. In some embodiments, the tailored formulations can be prepared in a laboratory. Taken together, the wellness App and dispenser are, in some embodiments, referred to as an apparatus for dispensing.

The below table 2 illustrates examples of dosing regimens.

In various embodiments, once a user takes a formulated dose, in some embodiments, the wellness App will have AI to assist with prompted voice or text instructions to complete the session with feedback. In some embodiments, questions such as How was the pain level today? How stressed were you today, from 1 to 10? Did you sleep well last night? can be asked. By this questioning and response session, in some embodiments, the loop is closed in that the user's state of being will be assessed, data will be entered, and this will enhance the accuracy of the platform and that can continue to add to the AI and Machine learning algorithm, improving responses, and formulations, as more information becomes available.

In some embodiments, once the user agrees and accepts, inputting the parameters or data, the dispensing of liquid, or solid herbal, Cannabis and/or pharmaceutical compounds for the user follow. This is customized dosage, a tailored Cannabis formulation, tailored for, in some embodiments, a one time serving for that user.

In various embodiments, personal data is entered via the wellness APP. Data collected, in some embodiments, includes for example electronic medical records (EMRs) or electronic patient records (EPRs), places where health information is securely captured and stored and is accessible only by health care networks, databases and other enterprise exchanges. Using the EMRs for a user, for example, the wellness APP can capture a range of data such as age, weight, sex, vital signs, medical history, medications, allergies, lab tests, physical & mental health profile, demographics and billing. In various embodiments, access and compiling of data in various embodiments described herein is, and can be kept, in compliance with general international data protection regulations such as HIPPA in the United States and in compliance with General Data Protection Regulation in the USA (HIPPA), Great Britain & EU.

In various embodiments, a method of providing tailored dosing of Cannabis formulations comprises first establishing a baseline dosage treatment plan. To achieve a baseline dosage treatment plan tailored to a user's needs, issues, what prompted them to seek treatment, are first considered, and entered into the wellness APP. The level of satisfaction with dosing on different days is assessed by user interplay with the wellness APP.

Based on a user's issues and needs, what prompted them to seek treatment, level of satisfaction with each dose given that day, the stages in the treatment process, total length of time the process took, and patient's level of pain during and after dosage treatment.

In some embodiments, each dosing can be calculated based on an algorithm whereby a ratio of THC:CBD is combined. Various ratios can be used. For example, it can be 1:1, 2:1, 3:1, 5:1, 10:1, 12:1, 15:1, or 20:1, all depending on the treatment plan and the therapy chosen. Persons of skill in the art would understand that these ratios are illustrative, and any ratio determined to achieve desired results can be used.

Example: for Anxiety, we may baseline the dose to be 10 MG THC:50 MG CBD to achieve a 1:5 ratio. The dosing treatment plan for subsequent days may include Titrating the dosing and ratio, usually would increase or decrease the ratios to achieve an optimal result for the user.

Our dosing therapy with treatments for chronic pain, cancer, chemotherapy-induced nausea and vomiting, anorexia and weight loss associated with HIV, epilepsy, spasticity, Tourette syndrome, irritable bowel syndrome, amyotrophic lateral sclerosis (Lou Gehrig disease), Huntington's disease, Parkinson's disease, dystonia, dementia, glaucoma, traumatic brain injury, drug addiction, anxiety, depression, sleep disorders, terminal illness, post traumatic stress disorder (PTSD), psychoses.

### Optimizing Dosage Treatment:

Proper dosage is the most important factor in achieving the therapeutic benefits/effects, while preventing the onset of adverse effects of Cannabis. The range of dosing could affect one person to the next is so different, that no one dose response can satisfy the user. The higher the dose may result in better therapeutic outcome and a higher probability of adverse effects. This is a monophonic dose-response correlation. Cannabis does not follow this typical response pattern of expected response.

Our findings indicate that with increase dose, there tends to be a stronger onset and effect, while after a certain level, any dosage increase will result in weaker therapeutic outcome and effects. There is also a direct correlation of higher incidence of unwanted side effects. That means, less could be more.

In various embodiments, the AI and dosage treatment plan formulated takes in consideration of this for each user. The systems, methods, and apparatus described herein can correlate vast information databases to ensure there is a match with all of the user needs, such as endocannabinoid system sensitivity and balance so that there is no overstimulation by high dosing. The various embodiments described herein take into account the evolving physiology of the user. For example, the cannabinoid receptor can degrade, hence a user can build tolerance. To achieve the optimal dosing, the algorithm incorporated in the wellness App finds the best therapy threshold or therapy window and achieve bi-directional effects of Cannabis as well as sensitization methods to reduce Cannabis dose while improving the effectiveness and benefits. In various embodiments, the data correlation takes into account synergies, compatibilities, and incompatibilities between different formulation components, to continue to tailor responsive and safe tailored formulations for users.

### Distro System

In some embodiments, the dispenser will auto-replenish with THC, CBD, herbs and any supplement as originally ordered by the user. Once the medicine, compound or herbs are low, the system can, in some embodiments, place an order alert for the replenishment. The cartridge, box or bottle of pre-mixed material can, in some embodiment, be delivered by mail, delivery service as appropriate by local Cannabis laws.

As used, in various embodiments described herein, AI can continually improve correlation of data treatment plans, dosage, other users' treatment successes or failures, what to avoid and how to best achieve the right dose for the user. As dosing evolves in accordance with various embodiments, the auto replenishment technology can update to reflect these changes in dosage formulations.

### EXAMPLES

Example: 1 pre-formulation CBD Gel Tablet is 5 MG and the THC gel tablet 1 MG. If the dosage calls for 10 MG of CBD to combine with 2 MG of THC Gel tablet, then the dispenser will give a total of 2 Gel Tabs of 5 MG CBD and 2 Gel Tabs of 1 MG THC for a total of 4 Gel Tablets per dose serving.

The liquid dispensing part will work similar in a similar way to the solid dispensing. The amount of CBD full spectrum water soluble powder and other herbal compounds will be mixed and served with the liquid or combined with THC for proper dosage ratio per serving. It can serve as little as 0.5 MG per dose to +100 MG per serving or any combination thereof.

The foregoing descriptions have been presented for purposes of illustration and description. Other changes, substitutions, and alterations are also possible without departing from the spirit and scope of this disclosure.

1. An apparatus for providing user tailored Cannabis formulations, comprising:
a wellness App on a communication device, for gathering user data, interfaced with an artificial intelligence (AI) system configured to cross reference one or more database of Cannabis compounds, herbal compounds, or combinations thereof;
a dispenser which comprises:
a housing structure which comprises a water tank and a mixing cell;
one or more cartridges for storing and mechanically distributing measured amounts of compound for dispensing that are in operable connection with the wellness App,
and a dispensing operating intelligent controller operably connected to the wellness App, and
a dispensing mechanism fluidly connected to the mixing cell and under the control of the dispensing operating intelligent controller.

2. The apparatus of claim 1, wherein the communication device is selected from the group consisting of smartphone, tablet, mobile internet device, laptop, wearable computer, computer, personal digital assistant and combinations thereof.

3. The apparatus of claim 1, wherein the AI system processor comprises a pharmaceutical dosage formula algorithm and is capable of evolving treatment and dosages to be tailored to a user.

4. The apparatus of claim 1, wherein the one or more database is a database selected from the group consisting CDRmed, UCLA Health, PubMed, The United Medical Language Systems (R) (UMLS), Metathesaurus (R), WebMD, Cannabis med, National Center for Biotechnology Information (NCBI), Clinical Trials Gov, Annals of Internal Medicine CannaMD, Americans for Safe Access, BMJ.org, American Cancer Society, Cannabis Community Care & Research, Eureka Alert, Medicinal Cannabis, Animal Health Studies Database and combinations thereof.

5. The apparatus of claim 4, wherein the one or more database comprises data for Cannabis compounds.

6. A system for tailoring Cannabis dosage formulations for a user,
comprising a wellness App which comprises an artificial intelligence connected software application for establishing formulations based on a user entered personal data and a database of Cannabis compounds, and
a dispenser.

7. The system of claim 6, wherein the wellness App is a smartphone APP.

8. The system of claim 6, wherein the wellness App is in communication with the dispenser using a communication interface.

9. The system of claim 8, wherein the communication interface is selected from NFC, WIFI or Bluetooth.

10. The system of claim 6, wherein the user entered personal data is stored on a computer readable medium.

11. A method for obtaining tailored Cannabis dosing formulations for a user, comprising:
collecting user information by having the user engage with and enter information into an interactive wellness APP;
accessing Cannabis compound databases;
establishing a baseline Cannabis dosage by correlating the Cannabis database to fit with the user information;
preparing a baseline Cannabis dosage comprising two or more Cannabis compounds in amounts that achieve the baseline Cannabis dosage;
observing user responses to the baseline Cannabis dosage to provide user response data, and
entering the user response data into the wellness APP to obtain a tailored Cannabis dose for the user.

12. The method of claim 11, wherein user information is selected from medical conditions, symptoms, pain levels, and prescription medications.

13. The method of claim 11, further comprising optimizing dosage treatment by entering user data after dosing with the tailored Cannabis dose and syncing the data with an artificial intelligence algorithm for optimizing the Cannabis dosing with data from Cannabis databases in real-time.

14. The method of claim 11, wherein optimizing dosage treatment comprises analyzing data relating to Cannabis dosage and human wellbeing and comparing with user information to achieve an optimized Cannabis formulation that provides human wellbeing.

15. The method of claim 14, wherein human wellbeing is physical wellbeing.

16. The method of claim 14, wherein human wellbeing is psychological wellbeing.

17. The method of claim 11, further comprising onset control for one or more cannabinoid.

18. The method of claim 11, further comprising dispensing tailored Cannabis dosing formulation to the user.

19. (canceled)

20. The method of claim 11, wherein the Cannabis compounds are selected from the group consisting of CBG, CGC, CBD, THC, CBGa, CBCa, CBDa, THCa and combinations thereof.

21. The method of claim 11, wherein the Cannabis compounds are selected from the group consisting of CBG, CGC, CBD, THC, CBGa, CBCa, CBDa, THCa, terpenes and combinations thereof.

22. A method of providing tailored Cannabis dosing formulations to a user, comprising:
collecting user information using an interactive wellness APP;
accessing Cannabis compound databases;
establishing a baseline Cannabis dosage by correlating the Cannabis database to fit with the user information;
preparing a baseline Cannabis dosage comprising two or more Cannabis compounds in amounts that achieve the baseline Cannabis dosage;
observing user responses to the baseline Cannabis dosage to provide user response data;
entering the user response data into the wellness APP to obtain a tailored Cannabis dose for the user, and
dispensing a tailored Cannabis dose to the user.

23. The method of claim 22, further comprising iteratively improving the tailored Cannabis dose by using an artificial intelligence algorithm that processes user information and changing databases in cycles of improvement by matching criteria.
